(19) United States
(12) Patent Application Publication
(10) Pub. No.: US 2010/0016353 A1
(43) Pub. Date: Jan. 21, 2010

(54) BENZOIMIDAZOLE DERIVATIVES USEFUL AS ANTIPROLIFERATIVE AGENTS

(76) Inventors: Kirk Russell Henne, Belmot, CA (US); John Charles Kath, LaMesa, CA (US); Ruby Anthea Szewc, High Bridge, NJ (US)

Correspondence Address: PHARMACIA CORPORATION;GLOBAL PATENT DEPARTMENT, POST OFFICE BOX 1027, ST. LOUIS, MO, 63006, US

(21) Appl. No.: 11/718,115

(22) PCT Filed: Oct. 3, 2005
(86) PCT No.: PCT/IB2005/003034

§ 371 Date: Jun. 2, 2009

Related U.S. Application Data
(60) Provisional application No. 60/616,978, filed on Oct. 7, 2004.

(51) Int. Cl. A61K 31/4709; C07D 401/14; A61P 35/00; A61P 35/02

(52) U.S. Cl. 514/314; 546/167

(57) ABSTRACT

The invention relates to substantially pure compounds of the formula (1) and to pharmaceutically acceptable salts, prodrugs and solvates thereof, wherein R, R¹, R², R³, R⁴, and R5 are as defined herein. The invention also relates to pharmaceutical compositions comprising the compounds of formula (1) and methods of treating abnormal cell growth, such as cancer, in mammals by administering such pharmaceutical formulations.

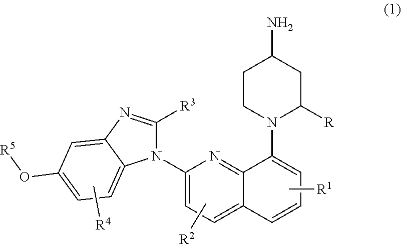

### BACKGROUND OF THE INVENTION

This invention relates to novel benzimidazole derivatives that are useful in the treatment of abnormal cell growth, such as cancer, in mammals. This invention also relates to a method of using such compounds in the treatment of abnormal cell growth in mammals, especially humans, and to pharmaceutical compositions containing such compounds.

It is known that a cell may become cancerous by virtue of the transformation of a portion of its DNA into an oncogene (i.e., a gene which, on activation, leads to the formation of malignant tumor cells). Many oncogenes encode proteins that are aberrant tyrosine kinases capable of causing cell transformation. Alternatively, the overexpression of a normal proto-oncogenic tyrosine kinase may also result in proliferative disorders, sometimes resulting in a malignant phenotype.

Receptor tyrosine kinases are enzymes which span the cell membrane and possess an extracellular binding domain for growth factors such as epidermal growth factor, a transmembrane domain, and an intracellular portion which functions as a kinase to phosphorylate specific tyrosine residues in proteins and hence to influence cell proliferation. Other receptor tyrosine kinases include c-erbB-2, c-met, tie-2, PDGFr, FGFr, and VEGFR. It is known that such kinases are frequently aberrantly expressed in common human cancers such as breast cancer, gastrointestinal cancer such as colon, rectal or stomach cancer, leukemia, and ovarian, bronchial or pancreatic cancer. It has also been shown that epidermal growth factor receptor (EGFR), which possesses tyrosine kinase activity, is mutated and/or overexpressed in many human cancers such as brain, lung, squamous cell, bladder, gastric, breast, head and neck, oesophageal, gynecological and thyroid tumors.

Accordingly, it has been recognized that inhibitors of receptor tyrosine kinases are useful as selective inhibitors of the growth of mammalian cancer cells. For example, erbstatin, a tyrosine kinase inhibitor, selectively attenuates the growth in athymic nude mice of a transplanted human mammary carcinoma that expresses epidermal growth factor receptor tyrosine kinase (EGFR) but is without effect on the growth of another carcinoma that does not express the EGF receptor. Thus, the compounds of the present invention, which are selective inhibitors of certain receptor tyrosine kinases, in particular PDGFr, are useful in the treatment of abnormal cell growth, in particular cancer, in mammals.

Various other compounds, such as styrene derivatives, have also been shown to possess tyrosine kinase inhibitory properties. More recently, five European patent publications, namely EP 0 566 226 A1 (published Oct. 20, 1993), EP 0 602 851 A1 (published Jun. 22, 1994), EP 0 635 507 A1 (published Jan. 25, 1995), EP 0 635 498 A1 (published Jan. 25, 1995), and EP 0 520 722 A1 (published Dec. 30, 1992), refer to certain bicyclic derivatives, in particular quinazoline derivatives, as possessing anti-cancer properties that result from their tyrosine kinase inhibitory properties. Also, World Patent Application WO 92/20642 (published Nov. 26, 1992), refers to certain bis-mono and bicyclic aryl and heteroaryl compounds as tyrosine kinase inhibitors that are useful in inhibiting abnormal cell proliferation. World Patent Applications WO96/16960 (published Jun. 6, 1996), WO 96/09294 (published Mar. 6, 1996), WO 97/30034 (published Aug. 21, 1997), WO 98/02434 (published Jan. 22, 1998), WO 98/02437 (published Jan. 22, 1998), and WO 98/02438 (published Jan. 22, 1998), also refer to substituted bicyclic heteroaromatic derivatives as tyrosine kinase inhibitors that are useful for the same purpose. Also see WO 99/16755, J. Med. Chem. 1998, 41, 5457-5465 and J. Med. Chem. 1999, 42, 2373-2382. World Patent Applications WO 04/020431 (published Mar. 11, 2004) and WO 01/40217 (published Jun. 7, 2001) disclose bicyclic heteroaromatic derivatives useful as tyrosine kinase inhibitors. Certain compounds disclosed in WO 04/020431 and WO 01/40217 are useful as precursors for the compounds described herein for preparing the claimed compounds using biotransformation within a mammal or by chemical synthesis. Each of the patents, patent applications, including published International, European and Great Britain applications, and scientific publications referred to in this patent application is incorporated herein by reference in its entirety.

### SUMMARY OF THE INVENTION

The present invention relates to a substantially pure compound of the formula 1

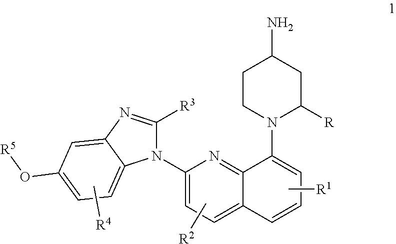

wherein R is selected from the group consisting of H, hydroxyl, oxo (═O), and glucuronide;

each R¹, R², R³, and R⁴ is independently selected from H, hydroxyl, and glucuronide; and

R⁵ is selected from the group consisting of:

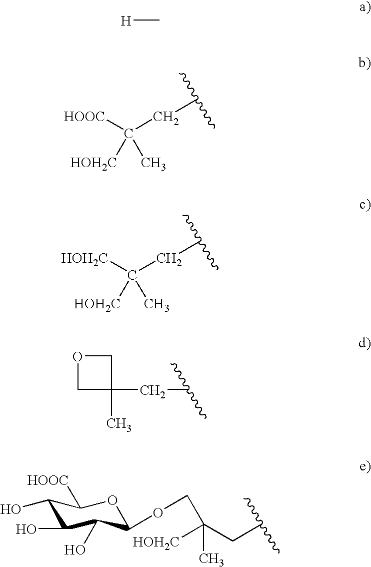

with the proviso that when R, R¹, R², R³, and R⁴ simultaneously are each hydrogen, then R⁵ cannot be

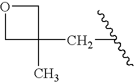

### or H,

or a pharmaceutically acceptable salt, prodrug, hydrate or solvate thereof.

Other embodiments of the invention include those compounds of formula 1, wherein R, R¹, R², R³ and R⁴ are hydrogen.

Other embodiments of the invention include those compounds of formula 1, wherein R, R¹, R², R³ and R⁴ are hydrogen and R⁵ is represented by the formula

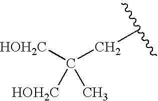

Other embodiments of the invention include those compounds of formula 1, wherein R, R¹, R², R³ and R⁴ are hydrogen and R⁵ is represented by the formula

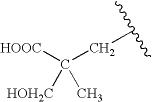

Other embodiments of the invention include those compounds of formula 1, wherein R, R¹, R², R³ and R⁴ are hydrogen and R⁵ is represented by the formula

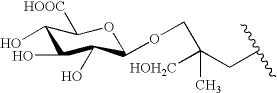

Other embodiments of the invention include those compounds wherein R, R², R³ and R⁴ are hydrogen and R¹ is hydroxyl.

Other embodiments of the invention include those compounds wherein R, R², R³ and R⁴ are hydrogen, R¹ is hydroxyl and R⁵ is represented by the formula

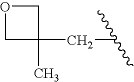

Other embodiments of the invention include those compounds of formula 1, wherein R, R¹, R² and R³ are hydrogen and R⁴ is hydroxyl.

Other embodiments of the invention include those compounds of formula 1, wherein R, R¹, R², and R³ are hydrogen, R⁴ is hydroxyl and R⁵ is represented by the formula

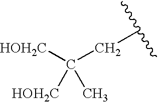

Other embodiments of the invention include those compounds of formula 1 wherein R, R², R³ and R⁴ are hydrogen and R¹ is glucuronide.

Other embodiments of the invention include those compounds of formula 1 wherein R, R², R³, and R⁴ are hydrogen, R¹ is glucuronide and R⁵ is

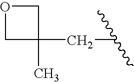

Preferred compounds include substantially pure compounds selected from the group consisting of:
2-{1-[8-(4-Amino-piperidin-1-yl)-quinolin-2-yl]-1H-benzoimidazol-5-yloxymethyl}-2-methyl-propane-1,3-diol;
3-{1-[8-(4-Amino-piperidin-1-yl)-quinolin-2-yl]-1H-benzoimidazol-5-yloxy}-2-hydroxymethyl-2-methyl-propionic acid;
4-Amino-1-{2-[5-(3-hydroxy-2-hydroxymethyl-2-methyl-propoxy)-benzoimidazol-1-yl]-quinolin-8-yl}-piperidin-2-one;
4-Amino-1-{2-[5-(3-methyl-oxetan-3-ylmethoxy)-benzoimidazol-1-yl]-quinolin-8-yl}-piperidin-2-ol;
8-(4-Amino-piperidin-1-yl)-2-[5-(3-methyl-oxetan-3-ylmethoxy)-benzoimidazol-1-yl]-quinolin-7-ol;
8-(4-Amino-piperidin-1-yl)-2-[5-(3-methyl-oxetan-3-ylmethoxy)-benzoimidazol-1-yl]-quinolin-6-ol;
8-(4-Amino-piperidin-1-yl)-2-[5-(3-methyl-oxetan-3-ylmethoxy)-benzoimidazol-1-yl]-quinolin-5-ol;
8-(4-Amino-piperidin-1-yl)-2-[5-(3-methyl-oxetan-3-ylmethoxy)-benzoimidazol-1-yl]-quinolin-4-ol;
8-(4-Amino-piperidin-1-yl)-2-[5-(3-methyl-oxetan-3-ylmethoxy)-benzoimidazol-1-yl]-quinolin-3-ol;
1-[8-(4-Amino-piperidin-1-yl)-quinolin-2-yl]-5-(3-methyl-oxetan-3-ylmethoxy)-1H-benzoimidazol-2-ol;
3-[8-(4-Amino-piperidin-1-yl)-quinolin-2-yl]-6-(3-methyl-oxetan-3-ylmethoxy)-3H-benzoimidazol-4-ol;
3-[8-(4-Amino-piperidin-1-yl)-quinolin-2-yl]-6-(3-methyl-oxetan-3-ylmethoxy)-3H-benzoimidazol-5-ol;
1-[8-(4-Amino-piperidin-1-yl)-quinolin-2-yl]-5-(3-methyl-oxetan-3-ylmethoxy)-1H-benzoimidazol-4-ol;
2-{1-[8-(4-Amino-piperidin-1-yl)-7-hydroxy-quinolin-2-yl]-1H-benzoimidazol-5-yloxymethyl}-2-methyl-propane-1,3-diol;
2-{1-[8-(4-Amino-piperidin-1-yl)-6-hydroxy-quinolin-2-yl]-1H-benzoimidazol-5-yloxymethyl}-2-methyl-propane-1,3-diol;
2-{1-[8-(4-Amino-piperidin-1-yl)-5-hydroxy-quinolin-2-yl]-1H-benzoimidazol-5-yloxymethyl}-2-methyl-propane-1,3-diol;
2-{1-[8-(4-Amino-piperidin-1-yl)-4-hydroxy-quinolin-2-yl]-1H-benzoimidazol-5-yloxymethyl}-2-methyl-propane-1,3-diol;
2-{1-[8-(4-Amino-piperidin-1-yl)-3-hydroxy-quinolin-2-yl]-1H-benzoimidazol-5-yloxymethyl}-2-methyl-propane-1,3-diol;
2-{1-[8-(4-Amino-piperidin-1-yl)-quinolin-2-yl]-2-hydroxy-1H-benzoimidazol-5-yloxymethyl}-2-methyl-propane-1,3-diol;
2-{1-[8-(4-Amino-piperidin-1-yl)-quinolin-2-yl]-4-hydroxy-1H-benzoimidazol-5-yloxymethyl}-2-methyl-propane-1,3-diol;
2-{1-[8-(4-Amino-piperidin-1-yl)-quinolin-2-yl]-6-hydroxy-1H-benzoimidazol-5-yloxymethyl}-2-methyl-propane-1,3-diol;
2-{-[8-(4-Amino-piperidin-1-yl)-quinolin-2-yl]-7-hydroxy-1H-benzoimidazol-5-yloxymethyl}-2-methyl-propane-1,3-diol;
2-{1-[8-(4-Amino-2-hydroxy-piperidin-1-yl)-quinolin-2-yl]-1H-benzoimidazol-5-yloxymethyl}-2-methyl-propane-1,3-diol;
6-(3-{1-[8-(4-Amino-piperidin-1-yl)-quinolin-2-yl]-1H-benzoimidazol-5-yloxy}-2-hydroxymethyl-2-methyl-propoxy)-3,4,5-trihydroxy-tetrahydro-pyran-2-carboxylic acid;
6-(4-Amino-1-{2-[5-(3-methyl-oxetan-3-ylmethoxy)-benzoimidazol-1-yl]-quinolin-8-yl}-piperidin-2-yloxy)-3,4,5-trihydroxy-tetrahydro-pyran-2-carboxylic acid;
6-{8-(4-Amino-piperidin-1-yl)-2-[5-(3-methyl-oxetan-3-ylmethoxy)-benzoimidazol-1-yl]-quinolin-7-yloxy}-3,4,5-trihydroxy-tetrahydro-pyran-2-carboxylic acid;
6-{8-(4-Amino-piperidin-1-yl)-2-[5-(3-methyl-oxetan-3-ylmethoxy)-benzoimidazol-1-yl]-quinolin-6-yloxy}-3,4,5-trihydroxy-tetrahydro-pyran-2-carboxylic acid;
6-{8-(4-Amino-piperidin-1-yl)-2-[5-(3-methyl-oxetan-3-ylmethoxy)-benzoimidazol-1-yl]-quinolin-5-yloxy}-3,4,5-trihydroxy-tetrahydro-pyran-2-carboxylic acid;
6-{8-(4-Amino-piperidin-1-yl)-2-[5-(3-methyl-oxetan-3-ylmethoxy)-benzoimidazol-1-yl]-quinolin-4-yloxy}-3,4,5-trihydroxy-tetrahydro-pyran-2-carboxylic acid;
6-{8-(4-Amino-piperidin-1-yl)-2-[5-(3-methyl-oxetan-3-ylmethoxy)-benzoimidazol-1-yl]-quinolin-3-yloxy}-3,4,5-trihydroxy-tetrahydro-pyran-2-carboxylic acid;
6-[1-[8-(4-Amino-piperidin-1-yl)-quinolin-2-yl]-5-(3-methyl-oxetan-3-ylmethoxy)-1H-benzoimidazol-2-yloxy]-3,4,5-trihydroxy-tetrahydro-pyran-2-carboxylic acid;
6-[1-[8-(4-Amino-piperidin-1-yl)-quinolin-2-yl]-5-(3-methyl-oxetan-3-ylmethoxy)-1H-benzoimidazol-4-yloxy]-3,4,5-trihydroxy-tetrahydro-pyran-2-carboxylic acid;
6-[3-[8-(4-Amino-piperidin-1-yl)-quinolin-2-yl]-6-(3-methyl-oxetan-3-ylmethoxy)-3H-benzoimidazol-5-yloxy]-3,4,5-trihydroxy-tetrahydro-pyran-2-carboxylic acid;
6-[3-[8-(4-Amino-piperidin-1-yl)-quinolin-2-yl]-6-(3-methyl-oxetan-3-ylmethoxy)-3H-benzoimidazol-4-yloxy]-3,4,5-trihydroxy-tetrahydro-pyran-2-carboxylic acid;

and the pharmaceutically acceptable salts, prodrugs, hydrates and solvates of the foregoing compounds.

Other embodiments of the invention include substantially pure compounds selected from the group consisting of
1-{2-[5-(3-Methyl-oxetan-3-ylmethoxy)-benzoimidazol-1-yl]-1-oxy-quinolin-8-yl}-piperidin-4-ylamine;
1-{2-[5-(3-Methyl-oxetan-3-ylmethoxy)-3-oxy-benzoimidazol-1-yl]-quinolin-8-yl}-piperidin-4-ylamine;
1-{2-[5-(3-Methyl-oxetan-3-ylmethoxy)-benzoimidazol-1-yl]-quinolin-8-yl}-1-oxy-piperidin-4-ylamine;
1-{2-[5-(3-Methyl-oxetan-3-ylmethoxy)-1-oxy-benzoimidazol-1-yl]-quinolin-8-yl}-piperidin-4-ylamine;

and the pharmaceutically acceptable salts, prodrugs, hydrates and solvates of the foregoing compounds.

In one preferred embodiment the compound of the present invention is the benzenesulfonate salt of any of the aforementioned compounds.

The invention also relates to a pharmaceutical composition for the treatment of abnormal cell growth in a mammal which comprises a therapeutically effective amount of a compound of formula 1, or a pharmaceutically acceptable salt, prodrug, solvate or hydrate thereof, and a pharmaceutically acceptable carrier. In one embodiment, said pharmaceutical composition is for the treatment of cancer such as brain, lung, squamous cell, bladder, gastric, pancreatic, breast, head, neck, renal, kidney, ovarian, prostate, colorectal, oesophageal, testicular, gynecological or thyroid cancer. In another embodiment, said pharmaceutical composition is for the treatment of a non-cancerous hyperproliferative disorder such as benign hyperplasia of the skin (e.g., psoriasis), restenosis, or prostate (e.g., benign prostatic hypertropy (BPH)).

The invention also relates to a pharmaceutical composition for the treatment of pancreatitis or kidney disease (including proliferative glomerulonephritis and diabetes-induced renal disease) in a mammal which comprises a therapeutically effective amount of a compound of formula 1, or a pharmaceutically acceptable salt, prodrug, solvate or hydrate thereof, and a pharmaceutically acceptable carrier.

The invention also relates to a pharmaceutical composition for the prevention of blastocyte implantation in a mammal that comprises a therapeutically effective amount of a compound of formula 1, or a pharmaceutically acceptable salt, prodrug, solvate or hydrate thereof, and a pharmaceutically acceptable carrier.

The invention also relates to a pharmaceutical composition for treating a disease related to vasculogenesis, restenosis, atherosclerosis or angiogenesis in a mammal which comprises a therapeutically effective amount of a compound of formula 1, or a pharmaceutically acceptable salt, prodrug, solvate or hydrate thereof, and a pharmaceutically acceptable carrier. In one embodiment, said pharmaceutical composition is for treating a disease selected from the group consisting of tumor angiogenesis, chronic inflammatory disease such as rheumatoid arthritis, atherosclerosis, skin diseases such as psoriasis, excema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

The invention also relates to a method for the treatment of abnormal cell growth in a mammal comprising administering to said mammal a pharmaceutical composition of the invention as described herein.

In one preferred embodiment of the present invention the abnormal cell growth is cancer.

In one embodiment of the present invention the cancer is selected from lung cancer, bone cancer, pancreatic cancer, gastric, skin cancer, cancer of the head or neck, cutaneous or intraocular melanoma, uterine cancer, ovarian cancer, gynecological, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, squamous cell, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain, pituitary adenoma, or a combination of one or more of the foregoing cancers.

In a preferred embodiment of the present invention the cancer is selected from the group consisting of brain, squamous cell, bladder, gastric, pancreatic, breast, head, neck, oesophageal, prostate, colorectal, lung, renal, kidney, ovarian, gynecological and thyroid cancer.

In a preferred embodiment of the present invention the cancer is selected from the group consisting of prostate, breast, lung, colon and ovarian cancer.

In another preferred embodiment of the present invention the cancer is selected from the group consisting of prostate, breast, and lung cancer.

In a more preferred embodiment the breast cancer is metastatic breast cancer.

In a more preferred embodiment the lung cancer is non-small cell lung cancer.

In another embodiment of the present invention the abnormal cell growth is non-cancerous.

In one embodiment of the present invention the non-cancerous abnormal cell growth is benign hyperplasia of the skin or prostate.

The invention also relates to a method for the treatment of vasculogenesis, restenosis, atherosclerosis or angiogenesis in a mammal comprising administering to said mammal a pharmaceutical composition according to the invention.

One preferred embodiment of the present invention is a method for treating a disease related to vasculogenesis or angiogenesis.

One embodiment of the present invention relates to a method for the treatment of a hyperproliferative disorder in a mammal which comprises administering to said mammal a pharmaceutical composition according to the invention in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

The invention also relates to a method of treating a hyperproliferative disorder in a mammal that comprises administering to said mammal a pharmaceutical composition according to the invention. In one embodiment, said method relates to the treatment of cancer such as brain, squamous cell, bladder, gastric, pancreatic, breast, head, neck, oesophageal, prostate, colorectal, lung, renal, kidney, ovarian, testicular, gynecological or thyroid cancer. In another embodiment, said method relates to the treatment of a non-cancerous hyperproliferative disorder such as benign hyperplasia of the skin (e.g., psoriasis), restenosis or prostate (e.g., benign prostatic hypertropy (BPH)).

The invention also relates to a method for the treatment of a hyperproliferative disorder in a mammal which comprises administering to said mammal a pharmaceutical composition according to the invention in combination with a therapeutically effective amount of an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

The invention also relates to a method of treating pancreatitis or kidney disease in a mammal which comprises administering to said mammal a pharmaceutical composition according to the invention.

The invention also relates to a method of preventing blastocyte implantation in a mammal which comprises administering to said mammal pharmaceutical composition according to the invention.

The invention also relates to a method of treating diseases related to vasculogenesis or angiogenesis in a mammal which comprises administering to said mammal a pharmaceutical composition according to the invention. In one embodiment, said method is for treating a disease selected from the group consisting of tumor angiogenesis, chronic inflammatory disease such as rheumatoid arthritis, atherosclerosis, skin diseases such as psoriasis, excema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

Patients that can be treated with pharmaceutical compositions comprising a compound of formula 1, and the pharmaceutically acceptable salts, prodrugs and hydrates of said compounds, according to the methods of this invention include, for example, patients that have been diagnosed as having psoriasis, restenosis, atherosclerosis, BPH, lung cancer, bone cancer, CMML, pancreatic cancer, skin cancer, cancer of the head and neck, cutaneous or intraocular melanoma, uterine cancer, ovarian cancer, rectal cancer, cancer of the anal region, stomach cancer, colon cancer, breast cancer, testicular, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphonas, cancer of the bladder, cancer of the kidney or ureter (e.g., renal cell carcinoma, carcinoma of the renal pelvis), or neoplasms of the central nervous system (e.g., primary CNS lymphona, spinal axis tumors, brain stem gliomas or pituitary adenomas).

This invention also relates to a pharmaceutical composition for inhibiting abnormal cell growth in a mammal which comprises a therapeutically effective amount of a compound of formula 1, or a pharmaceutically acceptable salt, hydrate, solvate or prodrug thereof, in combination with a pharmaceutically acceptable carrier and a chemotherapeutic agent, wherein the amounts of the compound, salt, hydrate, solvate, or prodrug, and of the chemotherapeutic agent are such that the pharmaceutical composition is effective in inhibiting abnormal cell growth. Many chemotherapeutic agents are presently known in the art. In one embodiment, the chemotherapeutic agent is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, and anti-hormones, e.g. anti-androgens.

This invention further relates to a method for inhibiting abnormal cell growth or treating a hyperproliferative disorder in a mammal which method comprises administering to the mammal a pharmaceutical composition according to the invention in combination with radiation therapy, wherein the amount of the compound, salt, hydrate, solvate or prodrug is in combination with the radiation therapy effective in inhibiting abnormal cell growth or treating the hyperproliferative disorder in the mammal. Techniques for administering radiation therapy are known in the art, and these techniques can be used in the combination therapy described herein. The administration of the compound of the invention in this combination therapy can be determined as described herein.

It is believed that the compounds of formula 1 can render abnormal cells more sensitive to treatment with radiation for purposes of killing and/or inhibiting the growth of such cells. Accordingly, this invention further relates to a method for sensitizing abnormal cells in a mammal to treatment with radiation which comprises administering to the mammal a pharmaceutical composition according to the invention in an amount and for a duration effective in sensitizing abnormal cells to treatment with radiation. The amount of the compound, salt, hydrate, prodrug or solvate in this method can be determined according to the means for ascertaining effective amounts of such compounds described herein.

This invention also relates to a pharmaceutical composition for inhibiting abnormal cell growth in a mammal which comprises (1) a compound of formula 1 or a pharmaceutically acceptable salt, prodrug, hydrate or solvate thereof, or an isotopically-labelled derivative thereof, (2) a pharmaceutically acceptable carrier, and (3) one or more substances selected from anti-angiogenesis agents, signal transduction inhibitors, and antiproliferative agents, and to methods of inhibiting abnormal cell growth in a mammal by administering such composition.

The present invention also relates to a method of determining if a patient has been administered 1-{2-[5-(3-Methyl-oxetan-3-ylmethoxy)-benzoimidazol-1-yl]-quinolin-8-yl}-piperidin-4-ylamine, the method comprising the step of determining if a plasma, urine, bile or fecal sample obtained from the patient shows the presence of a compound of formula 1.

The present invention also relates to a kit for the treatment of abnormal cell growth comprising a) a pharmaceutical composition comprising a therapeutically effective amount of a compound of formula 1 or a salt, solvate, hydrate or prodrug thereof and a pharmaceutically acceptable carrier; and b) instructions describing a method of using the pharmaceutical composition for treating the abnormal cell growth.

Anti-angiogenesis agents, such as MMP-2 (matrix-metalloproteinase 2) inhibitors, MMP-9 (matrix-metalloproteinase 9) inhibitors, and COX-II (cyclooxygenase II) inhibitors, can be used in conjunction with a compound of formula 1 and the pharmaceutical compositions described herein. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Publication 0818442 A2 (published Jan. 14, 1998), European Patent 1004578 (granted Feb. 25, 2004), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), WO 99/07675 (published Feb. 18, 1999), European Patent Application No. 99302232.1 (filed Mar. 25, 1999), Great Britain patent application number 9912961.1 (filed Jun. 3, 1999), European Patent Publication 1081137 (published Mar. 7, 2001), U.S. Pat. No. 5,863,949 (issued Jan. 26, 1999), U.S. Pat. No. 5,861,510 (issued Jan. 19, 1999), and European Patent Publication 780,386 (published Jun. 25, 1997). Preferred MMP-2 and MMP-9 inhibitors are those that have little or no activity inhibiting MMP-1. More preferred are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Some specific examples of MMP inhibitors useful in the present invention are AG-3340, RO 32-3555, RS 13-0830, and the compounds recited in the following list:
3-[[4-(4-fluoro-phenoxy)-benzenesulfonyl]-(1-hydroxycarbamoyl-cyclopentyl)-amino]-propionic acid;
3-exo-3-[4-(4-fluoro-phenoxy)-benzenesulfonylamino]-8-oxa-bicyclo[3.2.1]octane-3-carboxylic acid hydroxyamide;
(2R,3R) 1-[4-(2-chloro-4-fluoro-benzyloxy)-benzenesulfonyl]-3-hydroxy-3-methyl-piperidine-2-carboxylic acid hydroxyamide;
4-[4-(4-fluoro-phenoxy)-benzenesulfonylamino]-tetrahydro-pyran-4-carboxylic acid hydroxyamide;
3-[[4-(4-fluoro-phenoxy)-benzenesulfonyl]-(1-hydroxycarbamoyl-cyclobutyl)-amino]-propionic acid;
4-[4-(4-chloro-phenoxy)-benzenesulfonylamino]-tetrahydro-pyran-4-carboxylic acid hydroxyamide;
(R) 3-[4-(4-chloro-phenoxy)-benzenesulfonylamino]-tetrahydro-pyran-3-carboxylic acid hydroxyamide;
(2R,3R) 1-[4-(4-fluoro-2-methyl-benzyloxy)-benzenesulfonyl]-3-hydroxy-3-methyl-piperidine-2-carboxylic acid hydroxyamide;
3-[[4-(4-fluoro-phenoxy)-benzenesulfonyl]-(1-hydroxycarbamoyl-1-methyl-ethyl)-amino]-propionic acid;
3-[[4-(4-fluoro-phenoxy)-benzenesulfonyl]-(4-hydroxycarbamoyl-tetrahydro-pyran-4-yl)-amino]-propionic acid;
3-exo-3-[4-(4-chloro-phenoxy)-benzenesulfonylamino]-8-oxa-bicyclo[3.2.1]octane-3-carboxylic acid hydroxyamide;
3-endo-3-[4-(4-fluoro-phenoxy)-benzenesulfonylamino]-8-oxa-bicyclo[3.2.1]octane-3-carboxylic acid hydroxyamide; and
(R) 3-[4-(4-fluoro-phenoxy)-benzenesulfonylamino]-tetrahydro-furan-3-carboxylic acid hydroxyamide;

and pharmaceutically acceptable salts and solvates of said compounds.

Other anti-angiogenesis agents, including other COX-II inhibitors and other MMP inhibitors, can also be used in the present invention.

A compound of formula 1 and the pharmaceutical compositions according to the invention comprising a compound of formula 1 can also be used with signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors, such as VEGF receptors and molecules that can inhibit VEGF; and erbB2 receptor inhibitors, such as organic molecules or antibodies that bind to the erbB2 receptor, for example, HERCEPTIN™ (Genentech, Inc. of South San Francisco, Calif., USA).

EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998), and such substances can be used in the present invention as described herein. EGFR-inhibiting agents include, but are not limited to, the monoclonal antibodies C225, anti-EGFR 22Mab (ImClone Systems Incorporated of New York, N.Y., USA), and ABX-EGF (Abgenix antibody) the compounds ZD-1839 (AstraZeneca), BIBX-1382 (Boehringer Ingelheim), MDX-447 (Medarex Inc. of Annandale, N.J., USA), and OLX-103 (Merck & Co. of Whitehouse Station, N.J., USA), VRCTC-310 (Ventech Research) and EGF fusion toxin (Seragen Inc. of Hopkinton, Mass.). These and other EGFR-inhibiting agents can be used in the present invention.

VEGF inhibitors, for example SU-5416 and SU-6668 (Sugen Inc. of South San Francisco, Calif., USA), can also be combined with the compound of the present invention. VEGF inhibitors are described in, for example in WO 99/24440 (published May 20, 1999), WO 99/62890 (published Dec. 9, 1999), WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998). Other examples of some specific VEGF inhibitors useful in the present invention are IM862 (Cytran Inc. of Kirkland, Wash., USA); IMC-1C11 Imclone antibody, anti-VEGF monoclonal antibody of Genentech, Inc. of South San Francisco, Calif.; and angiozyme, a synthetic ribozyme from Ribozyme (Boulder, Colo.) and Chiron (Emeryville, Calif.). These and other VEGF inhibitors can be used in the present invention as described herein.

ErbB2 receptor inhibitors, such as GW-282974 (Glaxo Wellcome plc), and the monoclonal antibodies AR-209 (Aronex Pharmaceuticals Inc. of The Woodlands, Tex., USA) and 2B-1 (Chiron), can furthermore be combined with the compound of the invention, for example those indicated in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999). ErbB2 receptor inhibitors useful in the present invention are also described in U.S. Pat. No. 6,465,449 (issued Oct. 15, 2002) and in U.S. Pat. No. 6,284,764 (issued Sep. 4, 2001). The erbB2 receptor inhibitor compounds and substance described in the aforementioned PCT applications and U.S. patents, as well as other compounds and substances that inhibit the erbB2 receptor, can be used with the compound of the present invention in accordance with the present invention.

The compound and pharmaceutical composition of the invention can also be used with other agents useful in treating abnormal cell growth or cancer, including, but not limited to, agents capable of enhancing antitumor immune responses, such as CTLA4 (cytotoxic lymphocite antigen 4) antibodies, and other agents capable of blocking CTLA4; and anti-proliferative agents such as farnesyl protein transferase inhibitors, and αvβ3 inhibitors, such as the αvβ3 antibody Vitaxin, and αvβ5 inhibitors and the like. Specific CTLA4 antibodies that can be used in the present invention include those described in WO 00/37504 (published Jun. 29, 2000) and other CTLA4 antibodies known to those in the art.

The compounds and compositions of the invention can also furthermore be used in a palliative neo-adjuvant/adjuvant therapy in alleviating the symptoms associated with the diseases recited herein as well as the symptoms associated with abnormal cell growth. Such therapy can be a monotherapy or can be in a combination with chemotherapy and/or immunotherapy.

The invention also relates to a process for preparing the compound of formula 1 by incubating hepatocytes, preferably mammalian hepatocytes, and/or liver subcellular fractions such as microsomes, S9, or cytosol with 1-{2-[5-(3-Methyl-oxetan-3-ylmethoxy)-benzoimidazol-1-yl]-quinolin-8-yl}-piperidin-4-ylamine or a salt thereof under conditions and for a duration to produce a compound of formula 1 and isolating the resulting compound of formula 1.

The compound of formula 1 may also be prepared in vivo. For example, a mammal may be administered 1-{2-[5-(3-Methyl-oxetan-3-ylmethoxy)-benzoimidazol-1-yl]-quinolin-8-yl}-piperidin-4-ylamine and through biotransformation a compound of formula 1 is produced in the mammal.

The invention also relates to a process for preparing the compound of formula 1, comprising the step of preparing the compound synthetically. In one embodiment, the compound of formula 1 is prepared by treatment of 1-{2-[5-(3-Methyl-oxetan-3-ylmethoxy)-benzoimidazol-1-yl]-quinolin-8-yl}-piperidin-4-ylamine with an acid. In an another embodiment the compound of formula 1 is prepared according to the methods disclosed in World Patent Application WO 04/020431.

The terms “abnormal cell growth” and “hyperproliferative disorder” are used interchangeably in this application.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “oxo”, as used herein, unless otherwise indicated, means a double-bonded oxygen attachment (═O).

The term “hydroxyl”, as used herein, unless otherwise indicated, means hydroxyl (—OH).

The term “alkyl”, as used herein, unless otherwise indicated, includes saturated monovalent hydrocarbon radicals having straight, branched, or cyclic moieties (including fused and bridged bicyclic and spirocyclic moieties), or a combination of the foregoing moieties. For an alkyl group to have cyclic moieties, the group must have at least three carbon atoms.

The phrase “pharmaceutically acceptable salt(s)”, as used herein, unless otherwise indicated, includes salts of acidic or basic groups that may be present in the compounds of formula 1. The compounds of formula 1 that are basic in nature are capable of forming a wide variety of salts with various inorganic and organic acids. The acids that may be used to prepare pharmaceutically acceptable acid addition salts of such basic compounds of formula 1 are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phosphate/diphosphate, polygalacturonate, salicylate, stearate, subacetate, succinate, tannate, tartrate, teoclate, tosylate, triethiodode, and valerate salts. Since a single compound of the present invention may include more than one acidic or basic moieties, the compounds of the present invention may include mono, di or tri-salts in a single compound.

Those compounds of the present invention that are acidic in nature are capable of forming base salts with various pharmacologically acceptable cations. Examples of such salts include the alkali metal or alkaline earth metal salts and, particularly, the calcium, magnesium, sodium and potassium salts of the compounds of the present invention.

The present invention includes within its scope prodrugs of the compounds of formula 1 above. In general, such prodrugs will be functional derivatives of the compounds of formula 1 which are readily convertible in vivo into the required compound of formula 1. Conventional procedures for the selection and preparation of suitable prodrug derivatives are described, for example, in “Design of Prodrugs”, ed. H. Bundgaard, Elsevier, 1985.

A prodrug may be a pharmacologically inactive derivative of a biologically active substance (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulphate ester, or reduction or oxidation of a susceptible functionality.

The phrase “substantially pure”, as used herein, unless otherwise indicated, refers to the purity of chemical compounds wherein the compounds are preferably at least 90% pure, more preferably at least 95% pure, and most preferably at least 99% pure.

In another embodiment of the invention, the compounds of the claimed invention are purified and isolated.

As used herein, the notation

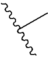

refers to a point of attachment. Thus the structure

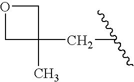

indicates that the point of attachment is the carbon atom of the CH₂ group.

The term “glucuronide” as used herein to refer to a substituent on another molecule is interchangeable with the structure

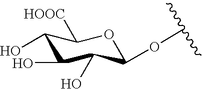

The compounds according to the invention have one or more asymmetric centers, and may accordingly exist both as enantiomers and as diastereoisomers. It is to be understood that all such isomers and mixtures thereof are encompassed within the scope of the present invention.

The compounds of formula 1 may also exist as tautomers. This invention relates to the use of all such tautomers and mixtures thereof. Furthermore, the invention concerns substantially pure compounds of the formula 1 in which the compound is present as a mixture of different enantiomers, diasteriomers or tautomers.

The subject invention also includes isotopically-labelled compounds, which are identical to those recited in formula 1 but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula 1 of this invention and prodrugs thereof can generally be prepared by carrying out the procedures disclosed in the Schemes and/or in the Examples and Preparations below and by substituting a readily available isotopically labelled reagent for a non-isotopically labelled reagent.

This invention also encompasses pharmaceutical compositions containing and methods of treating proliferative disorders, or abnormal cell growth, by administering prodrugs of compounds of the formula 1. Compounds of formula 1 having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula 1. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

### DETAILED DESCRIPTION OF THE INVENTION

General synthetic methods which may be referred to for preparing the compounds of the present invention are provided in U.S. Pat. No. 5,990,146 (issued Nov. 23, 1999)(Warner-Lambert Co.) and PCT published application numbers WO 99/16755 (published Apr. 8, 1999)(Merck & Co.), WO 04/020431 (published Mar. 11, 2004)(Pfizer, Inc.) and WO 01/40217 (published Jul. 7, 2001)(Pfizer, Inc.).

The compounds of the present invention may have asymmetric carbon atoms. Diasteromeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

The compounds of formula 1 are basic in nature and are capable of forming a wide variety of different salts with various inorganic and organic acids. Although such salts must be pharmaceutically acceptable for administration to animals, it is often desirable in practice to initially isolate the compound of formula 1 from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

The activity of the compounds of formula 1 may be determined by the following procedure.

General PGT Kinase ELISA Method

The following reagent and stock solutions are used:

| | |
|---|---|
| adenosine triphosphate (ATP) | Sigma, cat. # A-2383 |
| bovine serum albumin (BSA) | Sigma, cat. # A-3294 |
| Dulbecco's PBS (dPBS) | Gibco-BRL, cat. # 14190-136 |
| MaxiSorp plates | Nunc, cat. # 439454 |
| MgCl₂ | Sigma, cat. # M-1028 |
| Poly-Glu-Tyr (PGT) | Sigma, cat. #. P-0275 |
| TMB Micowell Substrate | Kirkegaard & Perry, cat. # 50-76-05 |
| Tween 20 | Sigma, cat. # P-1379 |
| HRP-PY54 antibody | OSI Pharmaceuticals, Inc. |

Phosphorylation Buffer (PB): 50 mM HEPES, pH 7.3, 125 mM NaCl, 24 mM MgCl₂;

Wash Buffer (WB): dPBS+0.1% Tween 20 (polyoxyethylene sorbitan); and

Blocking Buffer: 3% BSA, 0.05% Tween 20 in dPBS.

Assay Procedure:

(a) For plate coating, fill Nunc MaxiSorp plate with 100 μl per well of Poly-Glu-Tyr (PGT) diluted in dPBS (various concentrations). The plate is the incubated overnight at 37° C. The supernatant PGT is then disgarded, and the plates are washed 3× with Wash Buffer.

(b) The PDGF enzyme is then diluted in PB to an appropriate concentration, and 25 μl of this stock solution is added per well.

(c) ATP is then diluted (from 20 mM stock) to an appropriate concentration (0.5 nM-2 uM) with PB. The phosphorylation reaction is commenced by addition of 25 μl ATP solution to each well of the assay plate. Incubation is continued for about 10 minutes, with shaking at room temperature.

(d) The reaction is stopped by aspirating off the reaction mixture. The plate is then washed 4× with WB.

(e) The HRP-PY54 antibody is diluted to an appropriate concentration in blocking buffer. 50 μl of this solution is then added per well, followed by incubation for 25-35 minutes at room temperature. The antibody-containing solution is aspirated away, and the plate is again washed 4× with WB.

(f) The extent of reaction is determined by measurement of light absorbance at 450 nm. First, color is developed by addition of TMB solution, 50 μl per well, and the reaction is permitted to run until wells with positive signals achieve about 0.6-1.2 OD450 units. Color development is then stopped by addition of 50 μl per well of 0.09 M H2SO4. The background controls are wells without PGT, but with all other components included. As aforementioned, preferred signal is generally in the range of 0.6-1.2 OD units, with essentially no background.

The in vitro activity of the compounds of the present invention in inhibiting the PDGF receptor may be determined by the following procedure.

Inhibition of tyrosine kinase activity may be measured using a recombinant enzyme in an assay that measures the ability of compounds to inhibit the phosphorylation of the exogenous substrate, polyGluTyr (PGT, Sigma™, 4:1). The cytoplasmic domain of the human PDGFβ receptor (amino acids 559-1106) (Ishikawa, F., et al. Nature 338: 557-562, 1989) is expressed in Sf9 insect cells as a glutathione S-transferase (GST)-fusion protein using the baculovirus expression system. The protein is then purified from the lysates of these cells using glutathione agarose affinity columns.

The enzyme assay is performed in 96-well plates that are coated with the PGT substrate (0.625 μg PGT per well). Test compounds are diluted in dimethylsulfoxide (DMSO), and then added to the PGT plates so that the final concentration of DMSO in the assay is 1.6% (v/v). The recombinant enzyme is diluted in phosphorylation buffer (50 mM Hepes, pH 7.3, 125 mM NaCl, 24 mM MgCl₂). The reaction is initiated by the addition of ATP to a final concentration of 10 μM. After a 10 minute incubation at room temperature with shaking, the reaction is aspirated, and the plates are washed with wash buffer (PBS-containing 0.1% Tween-20). The amount of phosphorylated PGT is quantitated by incubation with a horseradish peroxidase(HRP)-conjugated PY-54 antibody (Transduction Labs), developing with TMB peroxidase (TMB is 3,3′,5,5′-tetramethylbenzidine), and detection on a BioRad™ Microplate reader at 450 nM. Inhibition of the kinase enzymatic activity by the test compound is detected as a reduced absorbance, and the concentration of the compound that is required to inhibit the signal by 50% (under the circumstances of the assay) is reported as the IC₅₀ value for the test compound.

To measure the ability of the compounds to inhibit PDGFRβ tyrosine kinase activity for the full length protein that exists in a cellular context, the porcine aortic endothelial (PAE) cells transfected with the human PDGFRβ (Westermark, Bengt, et. al., PNAS 87, pp 128-132, 1990) may be used. Cells are plated and allowed to attach to 96-well dishes in the same media (Ham's F12) with 10% FBS (fetal bovine serum) for 6-8 hours. The cells are washed, re-fed with serum depleted media, and allowed to incubate over night. Immediately prior to dosing with compound, the cells are re-fed with the serum depleted media. Test compounds, dissolved in DMSO, are diluted into the media (final DMSO concentration 0.5% (v/v)). At the end of a 10 minute incubation, PDGF-BB (100 ng/ml final) is added to the media for an 8 minute incubation. The cells are washed with Hepes buffered saline solution (HBSS) and lysed in 50 ul of HNTG buffer (20 mM Hepes, pH 7.5, 150 mM NaCl, 0.2% Triton™ X-100, 10% glycerol, plus 0.2 mM PMSF (phenymethylsulfonyl fluoride), 1 μg/ml pepstatin, 1 μg/ml leupeptin, 1 μg/ml aprotonin, 2 mM sodium pyrophosphate, 2 mM sodium orthovanadate) and then diluted with 50 ul of HG dilution buffer (20 mM Hepes, pH 7.5, 10% glycerol, 0.2 mM PMSF (phenymethylsulfonyl fluoride), 1 μg/ml pepstatin, 1 μg/ml leupeptin, 1 μg/ml aprotonin, 2 mM sodium pyrophosphate, 2 mM sodium orthovanadate). The extent of phosphorylation of PDGFRβ is measured using an ELISA assay. The 96-well Protein A coated plates are blocked with Superblock (Pierce) and coated with 0.5 μg per well anti-PDGFRβ P20 antibody (Santa Cruz, catalog number SC-339).

Any unbound antibody is washed off the plates prior to addition of the cell lysate. After a 2-hour room temperature incubation of the lysates (50 ul) with the PDGFRβ antibody, the PDGFRβ associated phosphotyrosine is quantitated by development with the HRP-conjugated PY-54 antibody and TMB, as described above. The ability of the compounds to inhibit the PDGF-BB stimulated autophosphorylation reaction by 50% under the conditions used, relative to PDGF-BB-stimulated controls, is reported as the IC₅₀ value for the test compound. The compounds of the present invention, including the examples recited below, generally have IC50 values using the foregoing procedure falling within the following range: 1-1000 nM.

Human liver cytosol incubations are conducted using commercially available cryopreserved cytosol (Tissue Transformation Technologies, 20 mg/mL protein, Lot #HHC-0255). Human liver cytosol is slowly thawed and diluted in 100 mM potassium phosphate buffer (pH 7.4) to a final protein concentration of 3.1 mg/mL and warmed to 37° C. Incubations are initiated with the addition of compound stock dissolved in methanol. Total methanol concentration is kept at or below 1%. After reaction initiation, incubation is gently mixed and a 0 min sample aliquot is collected and quenched in an equal volume of acetonitrile containing an internal standard. Subsequent time points are collected at 5, 10, 15 and 30 minutes and quenched in the same manner. Samples are centrifuged and the supernatants are analyzed by HPLC/MS/MS using the ratio of the peak area response of the analyte to that of the internal standard. A linear regression is fit to the data and half-lives are calculated from the slope of the line. The percent remaining calculations are performed using the half life of the fitted data. Control incubations are included to monitor interday variability and non-cytosolic mediated loss.

Administration of the compounds and pharmaceutical compositions of the present invention can be effected by any method that enables delivery of the compounds to the site of action. These methods include oral routes, intraduodenal routes, parenteral injection (including intravenous, subcutaneous, intramuscular, intravascular or infusion), intraocular, intraperitoneal, intravesicular, intravaginal, topical, and rectal administration.

Advantageously, the present invention also provides kits for use by a consumer for treating disease. The kits comprise a) a pharmaceutical composition comprising a therapeutically effective amount of compound of the present invention and a pharmaceutically acceptable carrier; and b) instructions describing a method of using the pharmaceutical composition for treating the specific disease.

A “kit” as used in the instant application includes a container for containing the separate unit dosage forms such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It maybe desirable to provide a written memory aid, where the written memory aid is of the type containing information and/or instructions for the physician, pharmacist or subject, e.g., in the form of numbers next to the tablets or capsules whereby the numbers correspond with the days of the regimen which the tablets or capsules so specified should be ingested or a card which contains the same type of information. Another example of such a memory aid is a calendar printed on the card e.g., as follows “First Week, Monday, Tuesday,” . . . etc. . . . “Second Week, Monday, Tuesday, . . . ” etc. Other variations of memory aids will be readily apparent. A “daily dose” can be a single tablet or capsule or several tablets or capsules to be taken on a given day.

Another specific embodiment of a kit is a dispenser designed to dispense the daily doses one at a time. Preferably, the dispenser is equipped with a memory-aid, so as to further facilitate compliance with the regimen. An example of such a memory-aid is a mechanical counter, which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds the patient when the next dose is to be taken.

In still another embodiment of the kits, the pharmaceutical composition may also comprise an additional compound that can be used in combination with a compound of the present invention, or the kit may comprise two pharmaceutical compositions: one containing a compound of the present invention and another containing an additional compound that can be used in combination with a compound of the present invention.

The amount of the active compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

The active compound may be applied as a sole therapy or may involve one or more other anti-tumour substances, for example those selected from, for example, mitotic inhibitors, for example vinblastine; alkylating agents, for example cis-platin, carboplatin and cyclophosphamide; anti-metabolites, for example 5-fluorouracil, cytosine arabinoside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

### Identification of the Metabolites of 1-{2-[5-(3-Methyl-oxetan-3-ylmethoxy)-benzoimidazol-1-yl]-quinolin-8-yl}-piperidin-4-ylamine in Selected Rat, Dog and Human Samples

Metabolite identification studies were performed on the parent compound, 1-{2-[5-(3-Methyl-oxetan-3-ylmethoxy)-benzoimidazol-1-yl]-quinolin-8-yl}-piperidin-4-ylamine, using in vitro and in vivo techniques in selected rat, dog and human samples. The parent compound is designated “P” in Scheme 1. In all species, the addition of water to the oxetane ring (M1) was the predominant metabolite of 1-{2-[5-(3-Methyl-oxetan-3-ylmethoxy)-benzoimidazol-1-yl]-quinolin-8-yl}-piperidin-4-ylamine in microsomal and hepatocyte incubations. The formation of M1 was NADPH dependent in rat and human but not mediated by cytochrome P₄₅₀ in dog. M1 was also the predominant metabolite in dog liver cytosolic incubations, but its formation is not inhibited by aldehyde oxidase (data not shown). Data from microsomal epoxide hydrolase inhibitor studies indicate that 1-{2-[5-(3-Methyl-oxetan-3-ylmethoxy)-benzoimidazol-1-yl]-quinolin-8-yl}-piperidin-4-ylamine is not a substrate for microsomal epoxide hydrolase. Subsequent oxidation of M1 on either the piperidine ring or one of the hydroxyl moieties formed via oxetane ring opening was observed in human liver microsomes and hepatocyte incubations (M2). No metabolites were observed in human liver cytosolic incubations. A mono-hydroxylated metabolite (M3) was found in both rat and dog liver microsomal incubations with oxidation occurring on the benzimidazole or quinoline ring systems or at the 2-position of the piperidine ring. M4, a secondary metabolite of the oxetane ring-opened metabolite M1, was observed in dog liver microsomes with oxidation occurring on the benzimidazole or quinoline ring structures. M1 also underwent further metabolism via glucuronidation in dog hepatocytes giving rise to M5. In addition, M3 appeared to be glucuronidated in rat and dog hepatocytes forming either M6 or M7; a positional isomer of M3 is believed to be the precursor of the M6 or M7 glucuronide.

M1 was found to be the major metabolite in dog plasma twenty minutes to one hour after 2.0 mg/kg intravenous administration of 1-{2-[5-(3-Methyl-oxetan-3-ylmethoxy)-benzoimidazol-1-yl]-quinolin-8-yl}-piperidin-4-ylamine. Due to the low levels of analyte in the plasma samples and relatively high background noise, a UV chromatogram depicting M1 formation could not be obtained. No other metabolites were detected in plasma. 1-{2-[5-(3-Methyl-oxetan-3-ylmethoxy)-benzoimidazol-1-yl]-quinolin-8-yl}-piperidin-4-ylamine appears to be excreted in bile after 5.0 mg/kg oral administration, in addition to M1, M2 and glucuronides M6 and M7. M8 was also observed in bile 2-4 hr post-dose; this metabolite was not observed in any of the in vitro matrices for all species studied. Due to a high level of matrix interference, no other metabolites were observed.

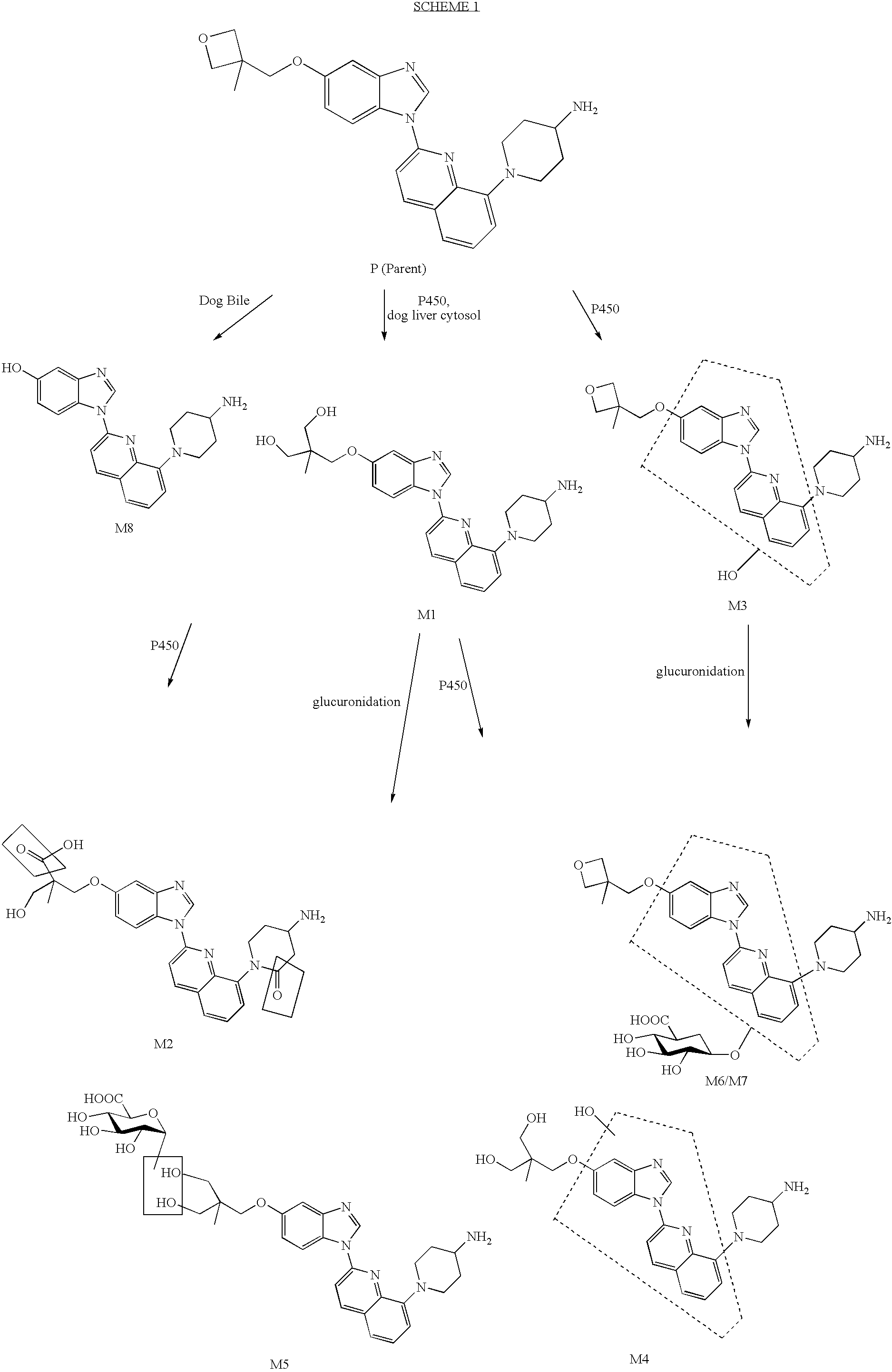

Materials and Methods

Microsomes: 1-{2-[5-(3-Methyl-oxetan-3-ylmethoxy)-benzoimidazol-1-yl]-quinolin-8-yl}-piperidin-4-ylamine (10 μM) was incubated in mouse, rat, dog, monkey and human liver microsomes and collected for analysis. 1-{2-[5-(3-Methyl-oxetan-3-ylmethoxy)-benzoimidazol-1-yl]-quinolin-8-yl}-piperidin-4-ylamine, (final concentration 10 mM) was incubated in mouse (0.45 mg/mL protein), rat (0.1.28 mg/mL protein), dog (0.1.18 mg/mL protein), monkey (0.42 mg/mL protein) and human liver microsomes (1.56 mg/mL protein) containing MgCl₂ (10 mM) and NADPH (1 mM) in potassium phosphate buffer (100 mM, pH 7.4) at 37° C. The total volume of each reaction mixture was 5 mL containing a total cytochrome P₄₅₀ concentration of 0.5 mM. Each mixture was preincubated for 10 minutes before addition of NADPH to initiate the reaction. The reaction was stopped one hour after addition of NADPH by adding two volumes of acetonitrile to the reaction mixture. Control incubations without NADPH were also conducted to assess metabolism not mediated by cytochrome P₄₅₀ in microsomes. Samples were prepared for analysis by centrifugation at 300 rpm to pellet the precipitated proteins and by evaporating the supernatant to dryness. The dried pellet samples were reconstituted in 600 μL 50/50 10 mM ammonium formate, pH 3.0/acetonitrile, and 10 μL aliquots were analyzed by liquid chromatography and mass spectroscopy (LC/MS/MS). 1-{2-[5-(2-Methoxy-ethoxy)-benzoimidazol-1-yl]-quinolin-8-yl}-piperidin-4-ylamine was incubated in dog liver microsomes under the same conditions as a positive control.

Hepatocytes: Rat, dog and human cryopreserved hepatocyte incubations were performed using 1-{2-[5-(3-Methyl-oxetan-3-ylmethoxy)-benzoimidazol-1-yl]-quinolin-8-yl}-piperidin-4-ylamine. Rat, dog and human cryopreserved hepatocytes were obtained from In Vitro Technologies, Baltimore, Md. and suspended in Williams E Media, available from Fisher-Scientific, to a final cell density of 1×106 viable cells/mL. 1-{2-[5-(3-Methyl-oxetan-3-ylmethoxy)-benzoimidazol-1-yl]-quinolin-8-yl}-piperidin-4-ylamine was incubated in these hepatocyte suspensions at a final concentration of 10 mM at 37° C. in a shaking water bath for up to four hours. A 5 mL aliquot was removed from each incubation mixture immediately and at four hours after addition of substrate. The samples were prepared for analysis by mixing with two volumes of acetonitrile followed by centrifugation to pellet precipitated proteins. The pellet samples were evaporated to dryness before analysis. Dried samples were reconstituted in 300 μL 50/50 10 mM ammonium formate, pH 3.0/acetonitrile, and 10 μL aliquots were analyzed by LC/MS/MS. 1-{2-[5-(2-Methoxy-ethoxy)-benzoimidazol-1-yl]-quinolin-8-yl}-piperidin-4-ylamine was incubated in dog hepatocytes under the same conditions as a positive control.

Cytosol: 1-{2-[5-(3-Methyl-oxetan-3-ylmethoxy)-benzoimidazol-1-yl]-quinolin-8-yl}-piperidin-4-ylamine (10 μM) was incubated and prepared for analysis. Cryopreserved human liver cytosol from Tissue Transformation Technologies Inc., Edison, N.J., (lot: HHC-0255, ˜20 mg/mL) and dog liver cytosol (˜35 mg/mL) were thawed on ice immediately before the incubation. The cytosol was diluted 10-fold in 100 mM potassium phosphate buffer (pH 7.4) before addition of 1-{2-[5-(3-Methyl-oxetan-3-ylmethoxy)-benzoimidazol-1-yl]-quinolin-8-yl}-piperidin-4-ylamine (final concentration 10 mM) for a final incubation volume of 2 mL. Samples were incubated for one hour in a 37° C. water bath, then quenched with two volumes of acetonitrile. Samples were vortexed and subsequently centrifuged at 3000 rpm. Samples were prepared for analysis by centrifugation at 3000 rpm to pellet the precipitated proteins and by evaporating the supernatant to dryness. The samples were reconstituted in 300 μL 50/50 10 mM ammonium formate, pH 3.0/acetonitrile. A 10 μL reconstituted aliquot was analyzed by LC/MS/MS. 1-{2-[5-(2-Methoxy-ethoxy)-benzoimidazol-1-yl]-quinolin-8-yl}-piperidin-4-ylamine was incubated in human and dog liver cytosol under the same conditions as a positive control.

Dog Plasma and Bile: Dog plasma was collected from beagle dogs following intravenous administration (2.0 mg/kg) of 1-{2-[5-(3-Methyl-oxetan-3-ylmethoxy)-benzoimidazol-1-yl]-quinolin-8-yl}-piperidin-4-ylamine. A 200 μL plasma sample from each dog was pooled for each timepoint. Three volumes of acetonitrile were added to the pooled plasma samples to precipitate proteins. Following centrifugation, the supernatant was recovered and dried at 37° C. under nitrogen and reconstituted in 150 μL 50/50 10 mM ammonium formate, pH 3.0/acetonitrile. A 20 μL aliquot was analyzed by LC/MS/MS.

Dog Bile: Bile was collected from beagle dogs following oral administration (5.0 mg/kg) of 1-{2-[5-(3-Methyl-oxetan-3-ylmethoxy)-benzoimidazol-1-yl]-quinolin-8-yl}-piperidin-4-ylamine. Aliquots (20 μL) from the 2-4 hr timepoints of various beagle dogs were subjected to LC/MS/MS analysis after centrifugation at 5000 rpm for 5 min to remove precipitated proteins and bile salts.

Synthetic Generation of M1: A 1 mg/mL solution of 1-{2-[5-(3-Methyl-oxetan-3-ylmethoxy)-benzoimidazol-1-yl]-quinolin-8-yl}-piperidin-4-ylamine was prepared in 0.25N HCl and allowed to sit overnight at room temperature. The reaction mixture was diluted 1:100 with 10 mM ammonium formate buffer, pH 3.0. A 100 μL sample was analyzed by LC/MS/MS to confirm generation of M1.

Sample Analysis

High Performance Liquid Chromatography

Separation of 1-{2-[5-(3-Methyl-oxetan-3-ylmethoxy)-benzoimidazol-1-yl]-quinolin-8-yl}-piperidin-4-ylamine and its metabolites in all samples was accomplished on a Zorbax SB-C18 reverse phase column (5 μm, 4.6×150 mm) at a flow rate of 1.0 mL/min. The mobile phase consisted of 10 mM ammonium formate, pH 3.0, (A) and acetonitrile (B). After injection of the sample, the column was washed with 10% B for 5 minutes followed by resolution of the metabolites with a linear gradient from 10% to 50% B over a 20-minute period. A 5-minute wash with 90% B followed by a 7-minute re-equilibration to 10% B was used between subsequent injections.

Mass Spectometry

All Samples were analyzed by a Finnigan TSQ7000 mass spectrometer operated in positive ion mode with in-line ultraviolet detection (Shimadzu, λ=254 nm). Ionspray voltage and capillary temperature were maintained at 4.5 kV and 350 degrees Celsius, respectively. Product ion spectra were obtained with CID of 35 V; further fragmentation was obtained by conducting product ion scans with source CID of 10 V. Neutral loss scans of 176 (for glucuronide detection) were conducted with CID of 30 V.

The metabolites were initially identified by molecular ion (Q1) scans of the sample mixture. Potential metabolites were characterized by fragmentation of the metabolite molecular ions and comparison of the resulting fragmentation patterns with the 1-{2-[5-(3-Methyl-oxetan-3-ylmethoxy)-benzoimidazol-1-yl]-quinolin-8-yl}-piperidin-4-ylamine product mass spectrum.

Identification of Metabolites

### 1-{2-[5-(3-Methyl-oxetan-3-ylmethoxy)-benzoimidazol-1-yl]-quinolin-8-yl}-piperidin-4-ylamine (Parent Drug)

The proposed metabolic pathway for 1-{2-[5-(3-Methyl-oxetan-3-ylmethoxy)-benzoimidazol-1-yl]-quinolin-8-yl}-piperidin-4-ylamine is presented in Scheme 1. The parent drug had an approximate retention time of 28.8 minutes on HPLC and showed a protonated molecular ion (M+H)⁺ at m/z 444.

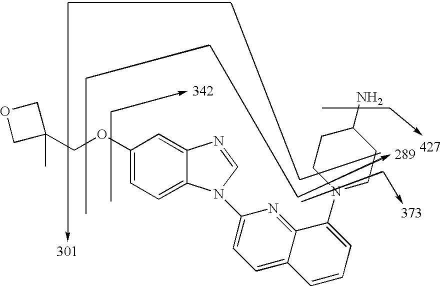

The MS spectra included major fragments at m/z 427, 373, and 301, and minor fragments at m/z 342 and 289. The ion at m/z 427 resulted from the loss of NH2. The diagnostic ions at m/z 373 and 301 resulted from cleavages of the piperidinyl ring and the oxetanyl group respectively. A CID spectrum generated with source CID of 10V; provided additional fragments at m/z 273, 261 and 155.

2-{1-[8-(4-Amino-piperidin-1-yl)-quinolin-2-yl]-1H-benzoimidazol-5-yloxymethyl}-2-methyl-propane-1,3-diol (Metabolite M1): M1 is the predominant metabolite of the parent compound in microsomal and hepatocyte incubations. The formation of M1 was NADPH dependent in rat and human and non-P450 mediated in dog. M1 was also the predominant metabolite in dog liver cytosolic incubations. M1 has an HPLC retention time of approximately 16.6 minutes. M1 had a protonated molecular ion (M+H)⁺ at m/z 462 and showed major fragment ions at m/z 445 (427+18), 391 (373+18), and 289 and minor fragment ions at m/z 373, 343, and 301. The CID spectrum generated with source CID showed additional fragment ions at m/z 261 and 155. The molecular ion was 18 amu more than the molecular ion of the parent demonstrating that the metabolite resulted from addition of water to the oxetane ring. The structure of this metabolite was confirmed by comparing the retention time and product ion spectrum of the metabolite with those of the acid-generated standard. In addition to generating M1, a chlorohydrin product (addition of HCl to the oxetane ring) was obtained when HCl was added to the parent compound.

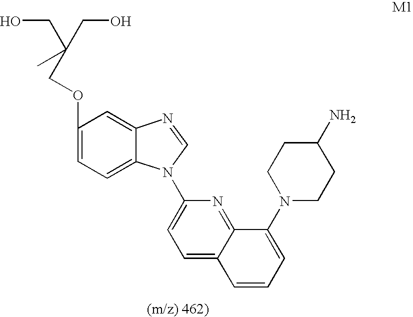

3-{1-[8-(4-Amino-piperidin-1-yl)-quinolin-2-yl]-1H-benzoimidazol-5-yloxy}-2-hydroxymethyl-2-methyl-propionic acid: 4-Amino-1-{2-[5-(3-hydroxy-2-hydroxymethyl-2-methyl-propoxy)-benzoimidazol-1-yl]-quinolin-8-yl}-piperidin-2-one (Metabolite M2): M2 was observed in human liver microsomes and hepatocyte incubations. No metabolites were observed in human liver cytosolic incubations. M2 has an HPLC retention time of approximately 16.9 minutes. M2 had a protonated molecular ion (M+H)⁺ at m/z 476 and showed major fragment ions at m/z 459 (427+32), 405 (373+32) and 357 and minor fragment ions at m/z 343 and 289. The CID spectrum generated with source CID showed additional fragment ions at m/z 301, 260 and 169 (155+14). The molecular ion was 32 amu more than the molecular ion of the parent compound, suggesting either further oxidation of M1 at the methylene group adjacent to the piperidine nitrogen or further oxidation of one of the hydroxyl groups to create one of two possible isomers of 3-{1-[8-(4-Amino-piperidin-1-yl)-quinolin-2-yl]-1H-benzoimidazol-5-yloxy}-2-hydroxymethyl-2-methyl-propionic acid.

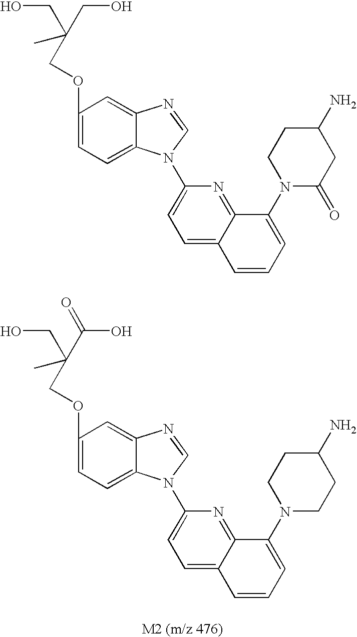

Metabolite M3: Metabolite M3 was found in both rat and dog liver microsomal incubations with oxidation occurring on the benzimidazole or quinoline ring systems or at the 2-position of the piperidine ring. M3 has an HPLC retention time of approximately 15.8 minutes. The M3 product ion mass spectrum of m/z 460 (M+H) showed fragment ions at m/z 443 (427+16), 389 (373+16) and 317 (301+16). The molecular ion was 16 amu higher than the molecular ion of the parent compound P suggesting hydroxylation of the rings, for example, 4-Amino-1-{2-[5-(3-methyl-oxetan-3-ylmethoxy)-benzoimidazol-1-yl]-quinolin-8-yl}-piperidin-2-ol or N-oxide formation, for example, 1-{2-[5-(3-Methyl-oxetan-3-ylmethoxy)-benzoimidazol-1-yl]-1-oxy-quinolin-8-yl}-piperidin-4-ylamine. The exact location of the oxidation site on the ring system could not be determined due to the limitations of mass spectrometry.

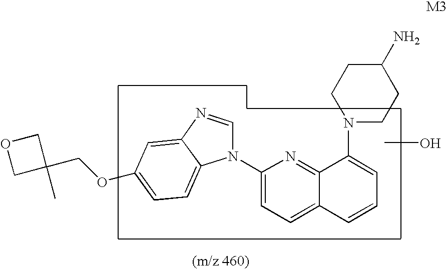

Metabolite M4: M4, a secondary metabolite of the oxetane ring-opened metabolite M4, found in dog liver microsomes, has an HPLC retention time of approximately 11.8 minutes. The M4 product ion mass spectrum of m/z 478 (M+H) showed fragment ions at m/z 461 (427+34), 407 (373+34), 359, and 317 (301+16). The molecular ion was 34 amu higher than the molecular ion of the parent compound illustrating further oxidation of M1 on the benzimidazole or quinoline ring systems, for example, 2-{1-[8-(4-Amino-piperidin-1-yl)-6-hydroxy-quinolin-2-yl]-1H-benzoimidazol-5-yloxymethyl}-2-methyl-propane-1,3-diol or the methylene adjacent to the piperidine nitrogen, that is 2-{1-[8-(4-Amino-2-hydroxy-piperidin-1-yl)-quinolin-2-yl]-1H-benzoimidazol-5-yloxymethyl}-2-methyl-propane-1,3-diol. Further elucidation of the oxidation site could not be determined due to limitations of mass spectrometry.

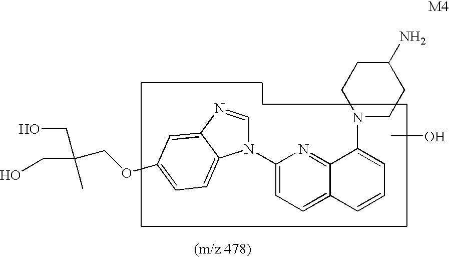

6-(3-{1-[8-(4-Amino-piperidin-1-yl)-quinolin-2-yl]-1H-benzoimidazol-5-yloxy}-2-hydroxymethyl-2-methyl-propoxy)-3,4,5-trihydroxy-tetrahydro-pyran-2-carboxylic acid (Metabolite M5): M5, found in dog hepatocytes, has an HPLC retention time of approximately 14.2 minutes. The M5 product ion mass spectrum of m/z 638 (M+H) showed fragment ions at m/z 462, 445 and 391. The molecular ion was 176 amu higher than the molecular ion of M1, demonstrating glucuronidation of M1 at one of the two propane diols.

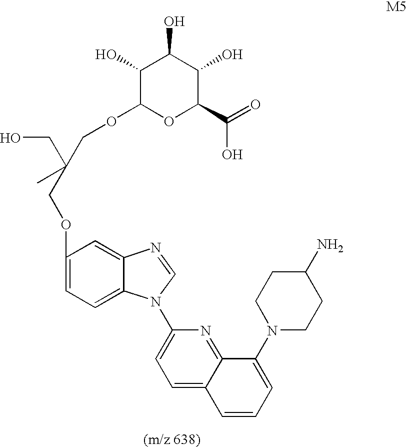

Metabolite M6: M6 is a glucuronide found in rat and dog hepatocytes. M6 has an approximate retention time of 5.2 minutes. The M6 product ion mass spectrum of m/z 636 (M+H) showed fragment ions at m/z 580, 564, 472, 460 and 389. The molecular ion was 176 amu higher than the molecular ion of M3, suggesting glucuronidation of M3.

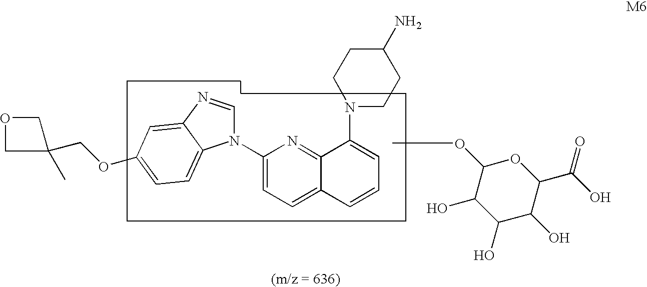

Metabolite M7: M7 is a glucuronide found in rat and dog hepatocytes. M7 has a HPLC retention time of 4.0 minutes. The M7 product ion mass spectrum of m/z 636 (M+H) showed fragment ions at m/z 565, 460, 442 and 389. The molecular ion was 176 amu higher than the molecular ion of M3, indicating glucuronidation of M3. This metabolite was chromatographically distinguishable from M6, suggesting that more than one isomer of M3 may be formed in some matrices.

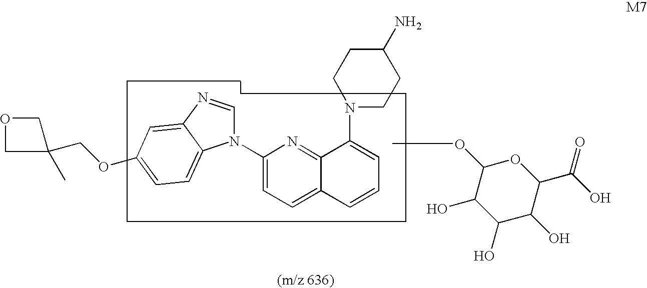

1-[8-(4-Amino-piperidin-1-yl)-quinolin-2-yl]-1H-benzoimidazol-5-ol Metabolite M8: M8 was found in dog bile 2-4 hours post-dose of the parent compound P. M8 was not observed in any of the in vitro matrices for all species studied. M8 has an HPLC retention time of approximately 15.5 minutes. The product ion mass spectrum of m/z 360 (M+H) showed fragment ions at m/z 343 and 289. The molecular ion was 58 amu lower than the molecular ion of the parent compound, demonstrating O-dealkylation of the parent compound P side chain. The structure of this metabolite was confirmed by comparing retention time and product ion spectrum of the metabolite with those of synthetic 1-[8-(4-Amino-piperidin-1-yl)-quinolin-2-yl]-1H-benzoimidazol-5-ol.

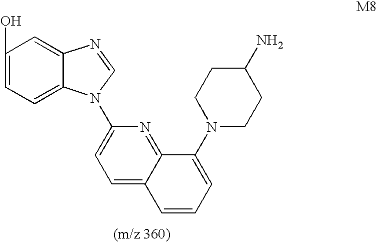

TABLE 1
Relative Abundance of 1-{2-[5-(3-METHYL-OXETAN-3-YLMETHOXY)-
BENZOIMIDAZOL-1-YL]-QUINOLIN-8-YL}-PIPERIDIN-4-YLAMINE
Metabolites in Preclinical Species and Human In Vitro and In Vivo Matrices

| | Metabolite | | | | | | | |
|---|---|---|---|---|---|---|---|---|
| | M1 | M2 | M3 | M4 | M5 | M6 | M7 | M8 |
| | Mass/charge | | | | | | | |
| | 462 | 476 | 460 | 478 | 638 | 636 | 636 | 360 |
| | Approx. Retention Time (min) | | | | | | | |
| | 16.6 | 16.9 | 15.8 | 11.8 | 14.2 | 5.2 | 4.0 | 15.5 |
| Human Liver Microsomes | +++ | +++ | − | − | na | na | na | − |
| HLM-NADPH ctrl | + | − | − | − | na | na | na | − |
| Rat Liver Microsomes | +++ | − | +++ | − | na | na | na | − |
| RLM-NADPH ctrl | + | − | − | − | na | na | na | − |
| Dog Liver Microsomes | +++ | + | +++ | + | na | na | na | − |
| DLM-NADPH ctrl | +++ | − | − | − | na | na | na | − |
| Human Hepatocytes | +++ | +++ | − | − | − | − | − | − |
| Rat Hepatocytes | +++ | +++ | − | − | − | +++ | + | − |
| Dog Hepatocytes | +++ | + | − | − | + | + | − | − |
| Human Liver Cytosol^{d} | − | − | − | − | − | − | − | − |
| Dog Liver Cytosol | +++ | − | − | − | − | − | − | − |
| Dog Plasma (IV)^{b} | +++ | − | − | − | − | − | − | − |
| Dog Bile (IV)^{c} | +++ | + | − | − | − | + | | + |

− metabolite not observed
+ minor metabolite
+++ major metabolite
na not applicable to microsomal system
^{a}Relative abundance was assessed by relative peak height on the UV chromatogram (major metabolites were designated as those peaks ≧ 10% of the base peak height)
^{b}Observed in 20 min, 40 min and 1 h samples
^{c}1-{2-[5-(3-Methyl-oxetan-3-ylmethoxy)-benzoimidazol-1-yl]-quinolin-8-yl}-piperidin-4-ylamine was also observed; only 2-4 hr samples were analyzed; cannot distinguish between M6 and M7 because of shift in retention time
^{d}No metabolites were observed in the human liver cytosol sample

1. A substantially pure compound of the formula 1 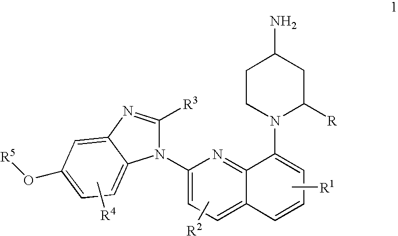
wherein R is selected from the group consisting of H, hydroxyl, oxo (═O), and glucuronide;
each R¹, R², R³, and R⁴ is independently selected from H, hydroxyl, and glucuronide; and
R⁵ is selected from the group consisting of: 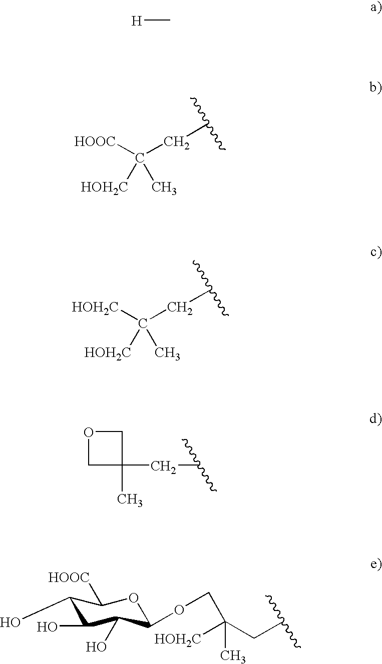
with the proviso that when R, R¹, R², R³, and R⁴ simultaneously are hydrogen, R⁵ cannot be 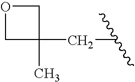
or H,
or a pharmaceutically acceptable salt, prodrug, hydrate or solvate thereof.

2. The compound according to claim 1, wherein R, R¹, R², R³ and R⁴ are hydrogen.

3. The compound according to claim 2, wherein R⁵ is represented by the formula

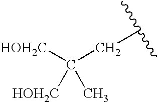

4. The compound according to claim 2, wherein R⁵ is represented by the formula

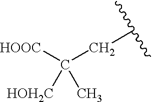

5. The compound according to claim 2, wherein R⁵ is represented by the formula

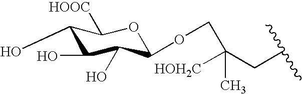

6. The compound according to claim 1, wherein R, R², R³ and R⁴ are hydrogen and R¹ is hydroxyl.

7. The compound according to claim 6, wherein R⁵ is represented by the formula

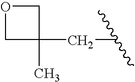

8. The compound according to claim 1, wherein R, R¹, R² and R³ are hydrogen and R⁴ is hydroxyl.

9. The compound according to claim 8, wherein R⁵ is represented by the formula

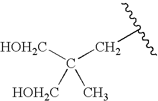

10. The compound according to claim 1, wherein R, R², R³ and R⁴ are hydrogen and R¹ is glucuronide.

11. The compound according to claim 10, wherein R⁵ is represented by the formula

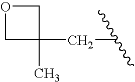

12. A substantially pure compound selected from the group consisting of:
2-{1-[8-(4-Amino-piperidin-1-yl)-quinolin-2-yl]-1H-benzoimidazol-5-yloxymethyl}-2-methyl-propane-1,3-diol;
3-{1-[8-(4-Amino-piperidin-1-yl)-quinolin-2-yl]-1H-benzoimidazol-5-yloxy}-2-hydroxymethyl-2-methyl-propionic acid;
4-Amino-1-{2-[5-(3-hydroxy-2-hydroxymethyl-2-methyl-propoxy)-benzoimidazol-1-yl]-quinolin-8-yl}-piperidin-2-one;
4-Amino-1-{2-[5-(3-methyl-oxetan-3-ylmethoxy)-benzoimidazol-1-yl]-quinolin-8-yl}-piperidin-2-ol;
8-(4-Amino-piperidin-1-yl)-2-[5-(3-methyl-oxetan-3-ylmethoxy)-benzoimidazol-1-yl]-quinolin-7-ol;
8-(4-Amino-piperidin-1-yl)-2-[5-(3-methyl-oxetan-3-ylmethoxy)-benzoimidazol-1-yl]-quinolin-6-ol;
8-(4-Amino-piperidin-1-yl)-2-[5-(3-methyl-oxetan-3-ylmethoxy)-benzoimidazol-1-yl]-quinolin-5-ol;
8-(4-Amino-piperidin-1-yl)-2-[5-(3-methyl-oxetan-3-ylmethoxy)-benzoimidazol-1-yl]-quinolin-4-ol;
8-(4-Amino-piperidin-1-yl)-2-[5-(3-methyl-oxetan-3-ylmethoxy)-benzoimidazol-1-yl]-quinolin-3-ol;
1-[8-(4-Amino-piperidin-1-yl)-quinolin-2-yl]-5-(3-methyl-oxetan-3-ylmethoxy)-1H-benzoimidazol-2-ol;
3-[8-(4-Amino-piperidin-1-yl)-quinolin-2-yl]-6-(3-methyl-oxetan-3-ylmethoxy)-3H-benzoimidazol-4-ol;
3-[8-(4-Amino-piperidin-1-yl)-quinolin-2-yl]-6-(3-methyl-oxetan-3-ylmethoxy)-3H-benzoimidazol-5-ol;
1-[8-(4-Amino-piperidin-1-yl)-quinolin-2-yl]-5-(3-methyl-oxetan-3-ylmethoxy)-1H-benzoimidazol-4-ol;
2-{1-[8-(4-Amino-piperidin-1-yl)-7-hydroxy-quinolin-2-yl]-1H-benzoimidazol-5-yloxymethyl}-2-methyl-propane-1,3-diol;
2-{1-[8-(4-Amino-piperidin-1-yl)-6-hydroxy-quinolin-2-yl]-1H-benzoimidazol-5-yloxymethyl}-2-methyl-propane-1,3-diol;
2-{1-[8-(4-Amino-piperidin-1-yl)-5-hydroxy-quinolin-2-yl]-1H-benzoimidazol-5-yloxymethyl}-2-methyl-propane-1,3-diol;
2-{1-[8-(4-Amino-piperidin-1-yl)-4-hydroxy-quinolin-2-yl]-1H-benzoimidazol-5-yloxymethyl}-2-methyl-propane-1,3-diol;
2-{1-[8-(4-Amino-piperidin-1-yl)-3-hydroxy-quinolin-2-yl]-1H-benzoimidazol-5-yloxymethyl}-2-methyl-propane-1,3-diol;
2-{1-[8-(4-Amino-piperidin-1-yl)-quinolin-2-yl]-2-hydroxy-1H-benzoimidazol-5-yloxymethyl}-2-methyl-propane-1,3-diol;
2-{1-[8-(4-Amino-piperidin-1-yl)-quinolin-2-yl]-4-hydroxy-1H-benzoimidazol-5-yloxymethyl}-2-methyl-propane-1,3-diol;
2-{1-[8-(4-Amino-piperidin-1-yl)-quinolin-2-yl]-6-hydroxy-1H-benzoimidazol-5-yloxymethyl}-2-methyl-propane-1,3-diol;
2-{1-[8-(4-Amino-piperidin-1-yl)-quinolin-2-yl]-7-hydroxy-1H-benzoimidazol-5-yloxymethyl}-2-methyl-propane-1,3-diol;
2-{1-[8-(4-Amino-2-hydroxy-piperidin-1-yl)-quinolin-2-yl]-1H-benzoimidazol-5-yloxymethyl}-2-methyl-propane-1,3-diol;
6-(3-{1-[8-(4-Amino-piperidin-1-yl)-quinolin-2-yl]-1H-benzoimidazol-5-yloxy}-2-hydroxymethyl-2-methyl-propoxy)-3,4,5-trihydroxy-tetrahydro-pyran-2-carboxylic acid;
6-(4-Amino-1-{2-[5-(3-methyl-oxetan-3-ylmethoxy)-benzoimidazol-1-yl]-quinolin-8-yl}-piperidin-2-yloxy)-3,4,5-trihydroxy-tetrahydro-pyran-2-carboxylic acid;
6-{8-(4-Amino-piperidin-1-yl)-2-[5-(3-methyl-oxetan-3-ylmethoxy)-benzoimidazol-1-yl]-quinolin-7-yloxy}-3,4,5-trihydroxy-tetrahydro-pyran-2-carboxylic acid;
6-{8-(4-Amino-piperidin-1-yl)-2-[5-(3-methyl-oxetan-3-ylmethoxy)-benzoimidazol-1-yl]-quinolin-6-yloxy}-3,4,5-trihydroxy-tetrahydro-pyran-2-carboxylic acid;
6-{8-(4-Amino-piperidin-1-yl)-2-[5-(3-methyl-oxetan-3-ylmethoxy)-benzoimidazol-1-yl]-quinolin-5-yloxy}-3,4,5-trihydroxy-tetrahydro-pyran-2-carboxylic acid;
6-{8-(4-Amino-piperidin-1-yl)-2-[5-(3-methyl-oxetan-3-ylmethoxy)-benzoimidazol-1-yl]-quinolin-4-yloxy}-3,4,5-trihydroxy-tetrahydro-pyran-2-carboxylic acid;
6-{8-(4-Amino-piperidin-1-yl)-2-[5-(3-methyl-oxetan-3-ylmethoxy)-benzoimidazol-1-yl]-quinolin-3-yloxy}-3,4,5-trihydroxy-tetrahydro-pyran-2-carboxylic acid;
6-[1-[8-(4-Amino-piperidin-1-yl)-quinolin-2-yl]-5-(3-methyl-oxetan-3-ylmethoxy)-1H-benzoimidazol-2-yloxy]-3,4,5-trihydroxy-tetrahydro-pyran-2-carboxylic acid;
6-[1-[8-(4-Amino-piperidin-1-yl)-quinolin-2-yl]-5-(3-methyl-oxetan-3-ylmethoxy)-1H-benzoimidazol-4-yloxy]-3,4,5-trihydroxy-tetrahydro-pyran-2-carboxylic acid;
6-[3-[8-(4-Amino-piperidin-1-yl)-quinolin-2-yl]-6-(3-methyl-oxetan-3-ylmethoxy)-3H-benzoimidazol-5-yloxy]-3,4,5-trihydroxy-tetrahydro-pyran-2-carboxylic acid;
6-[3-[8-(4-Amino-piperidin-1-yl)-quinolin-2-yl]-6-(3-methyl-oxetan-3-ylmethoxy)-3H-benzoimidazol-4-yloxy]-3,4,5-trihydroxy-tetrahydro-pyran-2-carboxylic acid;
1-{2-[5-(3-Methyl-oxetan-3-ylmethoxy)-benzoimidazol-1-yl]-1-oxy-quinolin-8-yl}-piperidin-4-ylamine;
1-{2-[5-(3-Methyl-oxetan-3-ylmethoxy)-3-oxy-benzoimidazol-1-yl]-quinolin-8-yl}-piperidin-4-ylamine;
1-{2-[5-(3-Methyl-oxetan-3-ylmethoxy)-benzoimidazol-1-yl]-quinolin-8-yl}-1-oxy-piperidin-4-ylamine;
1-{2-[5-(3-Methyl-oxetan-3-ylmethoxy)-1-oxy-benzoimidazol-1-yl]-quinolin-8-yl}-piperidin-4-ylamine; and the pharmaceutically acceptable salts, prodrugs, hydrates and solvates of the foregoing compounds.

13. A pharmaceutical composition for the treatment of abnormal cell growth in a mammal comprising a therapeutically effective amount of a compound of the formula 1 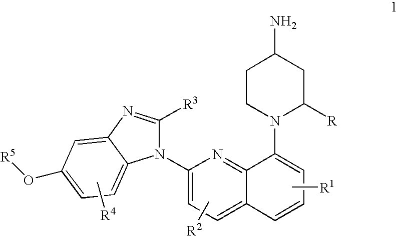
wherein R is selected from the group consisting of H, hydroxyl, oxo (═O), and glucuronide;
each R¹, R², R³, and R⁴ is independently selected from H, hydroxyl, and glucuronide; and
R⁵ is selected from the group consisting of: 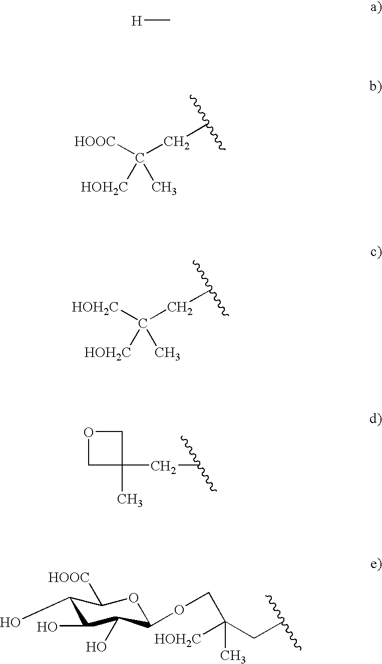
with the proviso that when R, R¹, R², R³, and R⁴ simultaneously are hydrogen, R⁵ cannot be 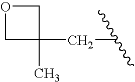
or H,
or a pharmaceutically acceptable salt, prodrug, hydrate or solvate thereof and a pharmaceutically acceptable carrier.

14. A method for the treatment of lung cancer, bone cancer, pancreatic cancer, gastric, skin cancer, cancer of the head or neck, cutaneous or intraocular melanoma, uterine cancer, ovarian cancer, gynecological, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, squamous cell, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain, pituitary adenoma, and a combination of one or more of the foregoing cancers in a mammal comprising administering to said mammal the pharmaceutical composition of claim 13.

15. A method for the treatment of a hyperproliferative disorder in a mammal which comprises administering to said mammal the pharmaceutical composition of claim 13 in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.
